(19) United States
(12) Patent Application Publication
(10) Pub. No.: US 2022/0249739 A1
(43) Pub. Date: Aug. 11, 2022

(54) BIOPOLYMER-BASED MENISCUS IMPLANT

(71) Applicant: Shu-Tung and Alice Li Foundation Inc., Franklin Lakes, NJ (US)

(72) Inventor: Shu-Tung Li, Franklin Lakes, NJ (US)

(73) Assignee: Shu-Tung and Alice Li Foundation Inc., Franklin Lakes, NJ (US)

(21) Appl. No.: 17/625,825

(22) PCT Filed: Aug. 4, 2020
(86) PCT No.: PCT/US2020/044824

§ 371 Date: Jan. 10, 2022

Related U.S. Application Data
(60) Provisional application No. 62/884,882, filed on Aug. 9, 2019.

(51) Int. Cl. A61L 27/26; A61L 27/38; A61L 27/36; A61L 27/54; A61L 27/56

(52) U.S. Cl. CPC A61L 27/26; A61L 27/3834; A61L 2430/06; A61L 27/54; A61L 27/56; A61L 27/3616

(57) ABSTRACT

The present invention relates to a glycosaminoglycan impregnated biopolymer-based scaffold implant for repairing and regeneration of damaged and diseased human menisci. The biopolymer can be any suitable natural or genetically engineered biopolymers. This scaffold implant has several advantages over prior art implants including higher biomechanical strength and lower surface friction. A method of making the scaffold implant is also disclosed.

### CROSS-REFERENCE TO RELATED APPLICATIONS

This application claims priority under 35 U.S.C. § 119(e) to U.S. Provisional Patent Application No. 62/884,882 filed Aug. 9, 2019. The foregoing application is incorporated by reference herein in its entirety.

### FIELD OF THE INVENTION

This invention relates to tissue engineering and regenerative medicine. Specifically, the present disclosure involves an improved meniscus implant for repairing injured or diseased meniscus.

### BACKGROUND

Menisci are two crescent shaped and slightly concaved wedges of fibrocartilage, situated between the femoral condyles and tibia plateau, providing crucial biomechanical functions to the knee joint. Damage to the meniscus will result in abnormal joint mechanics which is prelude to the development of osteoarthritis.

One of the major biopolymeric components in the extracellular matrix of meniscus is type I collagen which serves the structural supporting function of meniscus in the knee joint of the body. As type I collagen is homologous among all mammals, many implants made of type I collagen from animal sources are on the market today for tissue repair and regeneration (Li, 2012). Type I collagen in combination with a small amount of glycosaminoglycan (GAG) was used to make collagen implant for meniscus repair (U.S. Pat. Nos. 5,681,353; 5,735,903; 6,042,610; Li, et al., 2002, Type I collagen-based template for meniscus regeneration, eds., K-U Lewandrawski et al.). The implant was made with randomly oriented type I collagen GAG composite fibers. The product has been in clinical use both in Europe and in the US for more than 15 years with limited success, primarily due to insufficient mechanical strength to support the in vivo function as a temporary meniscus substitute during the period of healing. As a result, a long rehabilitation program is required to prevent re-tear of the implant pre-maturely. Even then, frequent tear or re-tear occurs that requires a second surgery to correct or replace the implant.

A synthetic polyurethane meniscus implant (Actifit^{R}, Orteq, Ltd., London, UK) was developed and marketed in Europe with limited usage. Therefore, only limited short term clinical data is available (Verdonk, et al., 2012, Am. J. Sports Med., 40:844-853). More recently, a polycarbonate-polyurethane meniscus implant (NuSurface^{R}, Active Implants, Inc., Memphis, Tenn. USA) was approved in Europe for the replacement of the whole meniscus. Again, the clinical outcome of the implant is unknown.

There is a need for a better meniscus implant for repairing injured or diseased meniscus.

### SUMMARY

This invention relates to tissue engineering and regenerative medicine. In one aspect, the invention features a scaffold implant for repairing injured or diseased human meniscus. The scaffold implant comprises fibers of a biopolymer impregnated with glycosaminoglycan. Greater than 50% (e.g., greater than 55%, 60%, or 70%) of the biopolymer fibers are oriented along the circumferential direction. The scaffold implant comprises a density from 0.10 g/cm³ to 0.40 g/cm³ (e.g., about 0.15 g/cm³ to about 0.30 g/cm³); a pore volume from 60% to 90%; and a surface friction coefficient from 0.05 to 1.0 (e.g., about 0.08 to about 0.8).

The biopolymer can be collagen, such as type I, type II and type III collagens. The collagen can be derived from human or animal, or made by genetic engineering technologies. The glycosaminoglycan can be hyaluronic acid, chondroitin sulfate, chitosan, or alginic acid, or a combination thereof. In one example, the glycosaminoglycan is hyaluronic acid. In that case, the weight percent of the hyaluronic acid in the scaffold implant can be in the range of about 1% to about 10% (e.g., 2% to about 7%). The molecular weight of the hyaluronic acid can range from about 0.1×10⁶ Daltons to about 3.0×10⁶ Daltons. In one example, the hyaluronic acids of various molecular weights can be impregnated into the scaffold implant via injection after the scaffold has been engineered.

The scaffold implant can be crosslinked by aldehyde-based molecules, such as formaldehyde or glycolaldehyde. The scaffold implant can further contain one or more bioactive elements. The bioactive elements can be autologous or allogenous. Examples of the bioactive elements include one or more selected from the group consisting of PRP, cells, and bioactive molecules. In one embodiment, the bioactive elements are autologous and include bioactive molecules or stem cells. In another embodiment, the bioactive elements are human recombinant bioactive molecules. In yet another embodiment, the bioactive elements are drugs.

In another aspect, the invention provides a method for making a hyaluronic acid impregnated collagen-based scaffold implant. The method comprises preparing a collagen dispersion; reconstituting collagen from the collagen dispersion into fibers; aligning the fibers onto a rotating mandrel to form aligned collagen fibers; placing the aligned collagen fibers in a mold of defined dimension; adding a weight to the fibers to dehydrate the fibers and form a dehydrated but still wet collagen scaffold matrix; freeze drying the dehydrated but still wet collagen scaffold matrix to obtain a freeze-dried scaffold matrix; crosslinking the freeze-dried scaffold matrix; sizing the scaffold matrix; and injecting hyaluronic acid into the scaffold matrix. The invention further provides a collagen-based meniscus scaffold implant prepared according to the method.

The invention further provides a method of treating or repairing a joint of a subject.

The method comprises providing the scaffold implant described above and delivering the scaffold implant to the joint.

To improve the drawbacks of the prior arts, and to provide the medical community with an improved meniscus implant for the repair of injured or diseased meniscus, the invention discloses a way to design and engineer a biopolymer type I collagen-based meniscus implant with the following improvements and advantages.

The type I collagen fibers in the implant are aligned along the biomechanical stress line in vivo to prevent pre-mature tear of the implant during initial healing period post-surgery.

The new implant significantly reduces the surface friction in contact with the femoral condyles by releasing hyaluronic acids of different molecular weight from the implant to protect the weight-bearing surface for long period of time during meniscus healing.

The density of the implant is balanced with mechanical strength, porosity and in vivo stability to support tissue regeneration and subsequent remodeling.

The new implant facilitates the rate of healing and reduces the rehabilitation time by further incorporating bioactive elements, cells and a combination of cells and bioactive elements at the point of surgery or by introducing bioactive elements, cells and a combination of cells and bioactive elements in in vitro systems prior to implantation.

The details of one or more embodiments of the invention are set forth in the description below. Other features, objectives, and advantages of the invention will be apparent from the description and from the claims.

### BRIEF DESCRIPTION OF THE DRAWINGS

FIGS. 1A, 1B, 1C, and 1D are diagrams showing a process of making a collagen-based scaffold meniscus implant.

FIG. 2 is an engineered prototype of human size medial meniscus scaffold.

FIGS. 3A and 3B show cross sectional pore structure taken by Scanning Electron Microscopy (SEM) where A is the section taken from the inner rim and B is the section taken from the outer rim of the meniscus prototype.

FIGS. 4A and 4B show fiber orientation along the circumference measured by fast Fourier transform program from ImageJ, where A shows the alignment fibers and B shows random fibers.

FIGS. 5A, 5B, 5C, 5D, 5E and 5F show results of fatigue test. The HA group (5B and 5E) shows less superficial layer disruption than the Saline group (5C and 5F), whereas the control group (5A and 5D) presents the normal surface.

FIG. 6 is table showing results of characterization studies.

### DETAIL DESCRIPTION OF THE INVENTION

A collagen-based scaffold meniscus implant was developed in the late 1990s for the repair of segmental defect of the meniscus. (U.S. Pat. Nos. 5,681,353; 5,735,903; 6,042,610; Li, et al., 2002). The implant was initially marketed in Europe and recently marketed in the US by Stryker. The clinical experience with this product has been mixed. Even though the implant supported new tissue growth, there are several drawbacks of using the product which are briefly summarized below.

The collagen fibers are randomly oriented in the product resulting in a low tensile strength along the circumferential stress direction. Also, the density of the product is not optimized to balance the pore size vs the overall strength. Since the pore volume (the empty space of the implant) is at about 85% (density 0.2 g/cm³), the overall mechanical properties to function as a meniscus scaffold is not optimal. Further, since the implant serves as a temporary meniscus substitute, the rate of resorption and the rate of new tissue deposition and subsequent tissue remodeling are not adequately balanced, leading to premature re-tear and failure. Finally, the friction coefficient is significantly higher than the surface of native meniscus, which can cause shear induced damages to the implant.

Due to the above drawbacks, a long rehabilitation program (3-6 months) is required for the product. As a result, many cases of re-tear surgeries were performed resulting from patients not in full compliance with the rehab protocol. In addition, there were many cases of incomplete regeneration due to loss of implant parts, resulting in smaller sizes of regenerated menisci, i.e., indicating mechanical insufficiency (Monllau, et al., 2011, Arthroscopy, 27:933-943).

An innovative new meniscus scaffold implant has been developed that can correct many drawbacks of the previous arts described above.

First, the collagen fibers are largely oriented along the direction of stress, minimizing the potential tear of the implant during the important healing period without sacrificing the suture retention (pull out) strength.

Second, the density of the scaffold implant is balanced without compromising other design requirements, such as the pore structure, to provide higher overall mechanical strength for in vivo stability.

Third, the surface in contact with femoral condyle will be continuously lubricated with hyaluronic acid, a key component of the synovial fluid in the body. Different sizes of hyaluronic acid molecules are impregnated into the implant for controlled release of the molecules to the surface at different rates, providing a prolong period of surface protection from potential shear stress induced surface damage.

Fourth, various bioactive elements can be incorporated in situ at the point of surgery to facilitate and enhance the rate of healing such that the rehab program can be more aggressively planned.

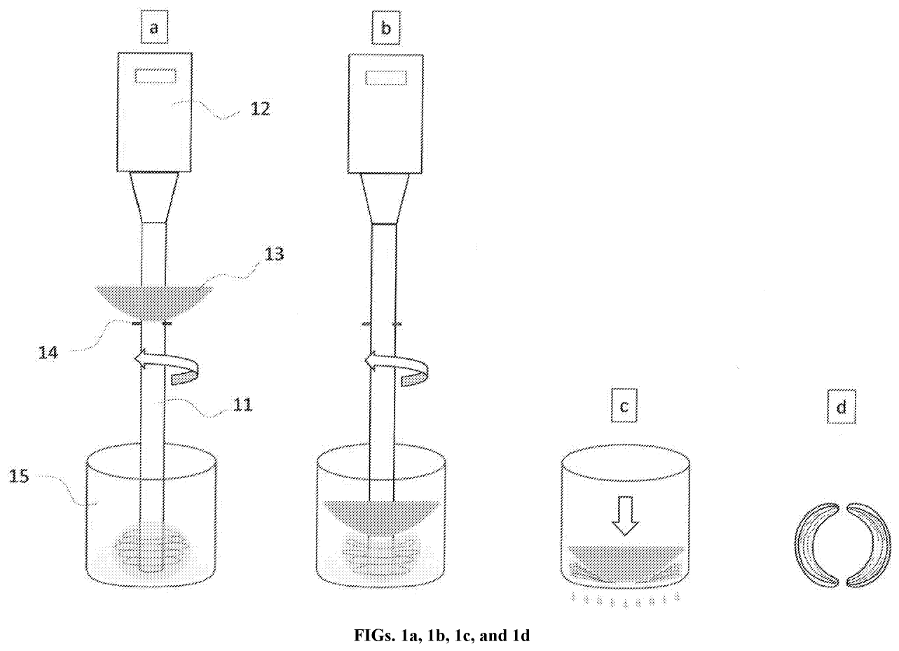

The following describes the method of engineering the collagen-based meniscus implant of the present invention. Please refer to the FIG. 1 in connection with the following steps.

Although various biopolymeric materials can be used to engineer the ECM scaffold implant, the inventor prefers the used of type I collagen. Type I collagen is the main component of the extracellular matrices and is homologous among all mammalian species. It is well accepted as a material for implant development. Purified type I collagen fibers were first prepared. Methods for collagen isolation and purification have been described previously (U.S. Pat. Nos. 5,681,353; 5,735,903; Li, et al., 2002). They are cited here as if set out in full. Briefly, tendon from bovine was used as the source for type I collagen. After cleaning and removing the extraneous tissues, the tendon was blocked, frozen and mechanically disintegrated into small pieces for ease of purification. The tendon was extracted in various solvents to remove the non-collagenous moieties, including various acidic and basic moieties (e.g., glycoproteins, GAGs, histones), lipids, DNAs, cell debris and various chemicals used in the process (e.g., acid, base salts, alcohols), rendering the purified collagen fiber implantable. The purified type I collagen was stored in the freeze-dried form.

A typical example of engineering a collagen scaffold implant is described below. A fixed weight of purified collagen fibers prepared above was first suspended in a fixed volume of 0.07M lactic acid for a final collagen content of 0.7% (w/v), pH 2.3 overnight at 4° C. and subsequently homogenized the suspension to reduce the fiber size to fibrils for uniform dispersion using a commercial homogenizer (Silverson, East Longmeadow, Mass). pH of aliquots of the dispersion, containing a fixed amount of collagen to form a final human size implant with an average density in the range of 0.10-0.40 g/cm³, were then adjusted to the isoelectric point of collagen (˜pH 5) with 1M NH₄OH to reconstitute the fibers. Upon de-air to remove the trapped air within the reconstituted fibers, the fibers were wrapped around a rotating stainless-steel mandrel covered with a PTFE tubing (OD 2.0 cm) at a rotating speed of 1-50 RPM to align the fibers (member 12 of FIG. 1). The hydrated fibers were partially dehydrated using a glass plate to remove the excess solution while the mandrel is rotating at a slower speed of about 1-50 RPM, forming a thick fiber matrix of aligned fibers with a wall thickness about 1.5 cm.

The partially dehydrated collagen fibers on the mandrel were then placed at the center of the molding element (15 of FIG. 1a). The weight element (14 in FIG. 1a) was then released from the top of the mandrel and slowly slid down to the top of the collagen fibers (FIG. 1b) and the mandrel was removed. As the base of the housing element was permeable to liquid, the weight element would continue to dehydrate the collagen fibers until it hit the fixed height, forming a disk-like dense matrix with defined density (FIG. 1c).

The significantly dehydrated but still wet collagen fiber scaffold disk was then freeze dried in a commercial freeze dryer (Virtis, Gardiner, N.Y.). The freeze-dried scaffold disk with circumferentially oriented fibers was then chemically crosslinked with vapor of a low molecular weight aldehyde compound (e.g., formaldehyde or glycol-aldehyde), generated from the solution at a concentration of 1-3% at room temperature in a cross-linking tank. Formaldehyde has been the most common chemical used to crosslink the porous collagen-based products. Many collagen products on the market today are crosslinked with formaldehyde. For porous collagen scaffold implant, vapor crosslinking is particularly preferred as vapor crosslinking can preserve the three-dimensional structure of the design. Formaldehyde has the advantage over other chemicals in that it is a small molecule with high vapor pressure at room temperature for crosslinking proteins such as collagen to maintain the three-dimensional size, shape and dimension of the original design. The disadvantage of formaldehyde crosslinking is that the residual formaldehyde must be removed after crosslinking since it is toxic to cells. Even though most of the formaldehyde is removed by water rinse and is acceptable for implantation, in vitro cell culture studies (a static culture system) often showed residual cell-toxicity effect. Thus, in in vitro cell culture studies, one should eliminate any potential side effects that can affect the cell behavior.

Like formaldehyde, glycolaldehyde can also be used as a vapor crosslinker, so the shape and form of the implant can be maintained. Glycolaldehyde has the advantage over formaldehyde in that glycol-aldehyde is much less cytotoxic than formaldehyde. The residual glycolaldehyde molecules do not produce any significant cytotoxicity and is a better crosslinking agent for porous collagen-based scaffold for in vitro cell culture studies. The crosslinked fiber disk was then cut in the middle to form two individual meniscus implants (FIG. 1D).

To reduce the surface friction, hyaluronic acid (HA), a major component of the synovial fluid, was applied to the interstitial space post engineering described above. The molecular weights of HA used ranged from about 0.1×10⁶ to about 3×10⁶ Daltons. 50 μl of 0.5-2% HA (w/v) was first loaded into a 30G needle syringe. The needle was inserted into a fixed position from the back (the thick side) of the meniscus implant and the HA was injected into the interstitial (intrafibrillar) space. This procedure was repeated 3-5 times at a fixed distance (about 3 mm apart) along the circumferential position and at a height from the bottom of about 2-3 mm. The HA impregnated implant was dried. Each molecular weight HA was applied to the meniscus implant to provide a prolonged release based on different diffusion rates of the molecules. The total amount of HA loaded was about 1-10% of the weight of the meniscus implant, preferably from 2-7%.

The improvement of surface friction is tested with a device constructed in house to simulate the knee joint mechanics. A pendulum is attached to a rotational disk which is linked to a sample housing unit that is closely associated with the rotational disk for the frictional testing. A weight element is attached to the sample housing so as that a weight can be applied to the sample which in turn transmitted to the rotational disk. A fixed weight is applied to the rotational disk and the pendulum is set to swing at a fixed angle which initiates the rotational disk to rotate on the surface of the sample. The system is immersed in a water chamber to allow the HA to diffuse to the surface to improve the surface frictional property. The number of pendulum swings within a defined angle and time is recorded. The scaffold with and without the HA as well as the native bovine meniscus is tested, and the data are compared.

To gain some insight on the effect of HA on the surface properties, the inventor conducted a preliminary study of immersing the collagen implant samples in a solution and tested the surface friction with and without the presence of HA.

A modified system of the above is used for durability testing. The duration of time with a fixed weight and RPM is tested. Samples with or without hyaluronic acid were examined by light microscopy or by SEM at the conclusion of the testing.

To facilitate the speed of healing, autologous bioactive elements or the like can be applied to the scaffold implant at the point of surgical implantation. This can be done via an injection technique. Autologous bioactive elements that can be used include platelet rich plasma (PRP), isolated from patient's blood, bioactive components isolated from patient's bone marrow, recombinant human growth factors, various bioactive macromolecules, differentiated cells and stem cells. Other methods of incorporation of bioactive elements into the scaffold implant include soaking the scaffold with bioactive element solutions and culturing the scaffold with various types of cells prior to implantation.

The above described various scaffold implants can be used to guide the new tissue regeneration of segmental defects in injured or diseased meniscus. It is important that the vascular portion of the defect is preserved and maintained such that autogenous cells and blood vessels can be infiltrated into the porous implant post-surgery during the healing period.

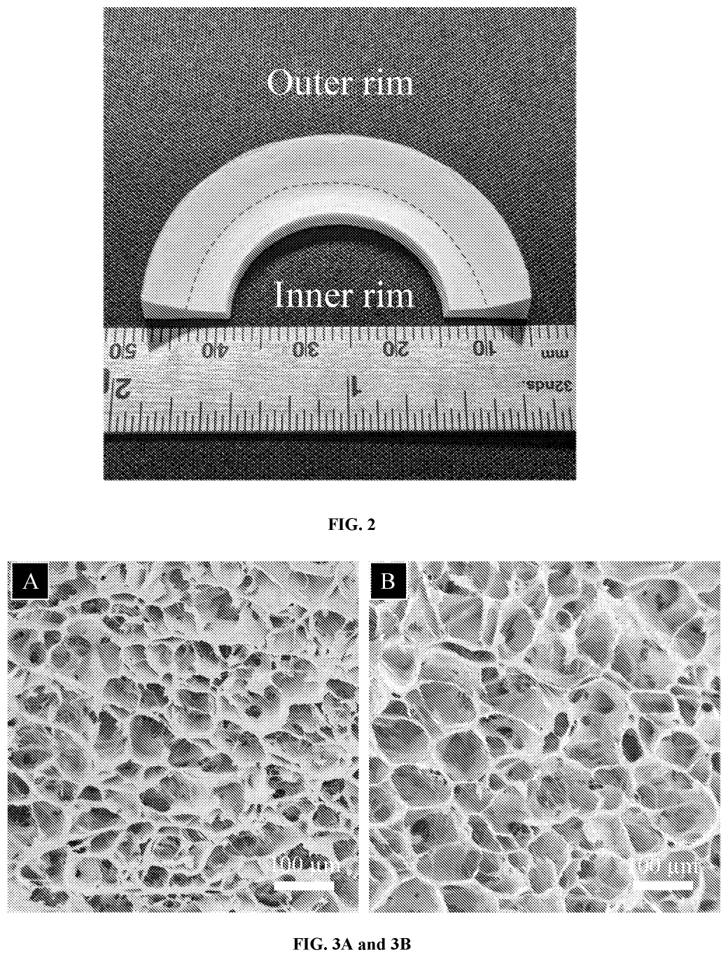

The following characterization studies were performed on the type I collagen fiber-based scaffold implant. In the characterization studies, the meniscus was divided into two regions, the outer ½ and inner ½ (see FIG. 2) as the two regions were subjected to different mechanical forces and had different healing characteristics. The results are summarized in Table 1.

### Density

A meniscus scaffold implant was dried in a desiccator under P₂O₅ overnight and weighed to obtain the dry weight of the scaffold matrix. The dry scaffold was then immersed in 100 ml of water in a beaker for 10 minutes. The surface water was removed with a lint free cloth. The hydrated scaffold was then weighed. The volume of the scaffold was taken as the sum of the volume occupied by the water and the volume occupied by the collagen. The density was calculated as the ratio of the dry weight of the scaffold and the total volume of the scaffold in g/cm³, taking the density of collagen to be 1.41 g/cm³ (Noda H., 1972 partial specific volume of collagen, J. Biochem., 71:699-703). The average density of the meniscus scaffold implant was in the range from 0.1 g/cm³ to 0.4 g/cm³.

### Pore Sizes and Pore Volume

The pore sizes were determined from the SEM micrographs at Stevens Institute of Technology via a contract service agreement. Two cross sections, radial and horizontal, were used. Pore size of each pore was determined as the maximum distance across a pore. The range of pore size was between 60 μm to 130 μm.

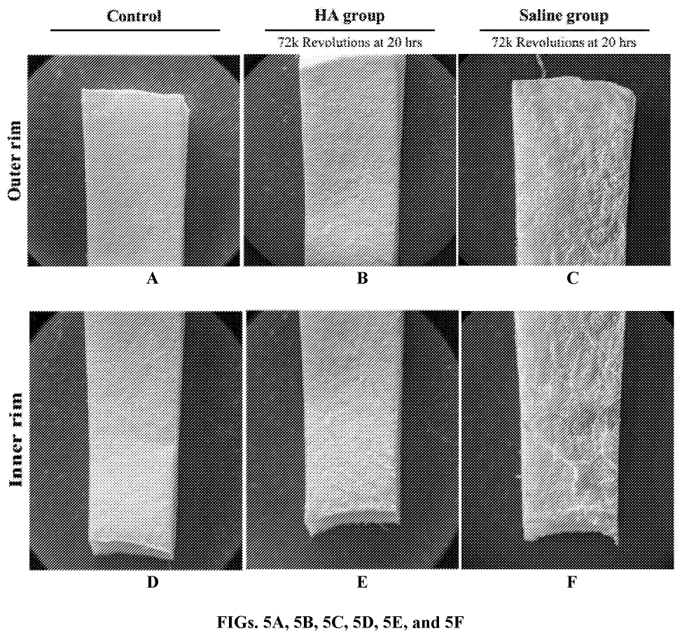

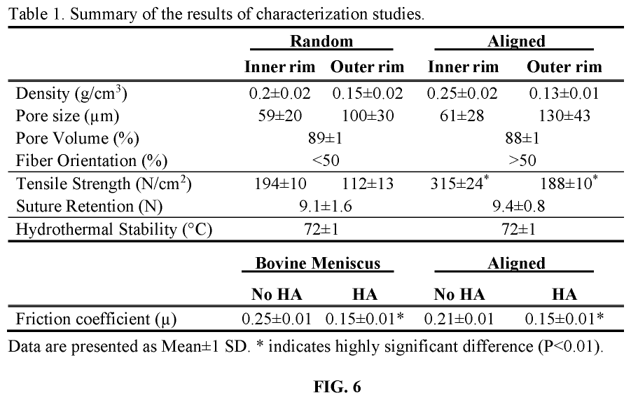

The percent (%) pore volume was defined as the empty space (wet weight minus volume of collagen) divided by the total volume. The pore volume of the scaffold fell within 60% to 90% (see FIG. 5).

### Fiber Orientation

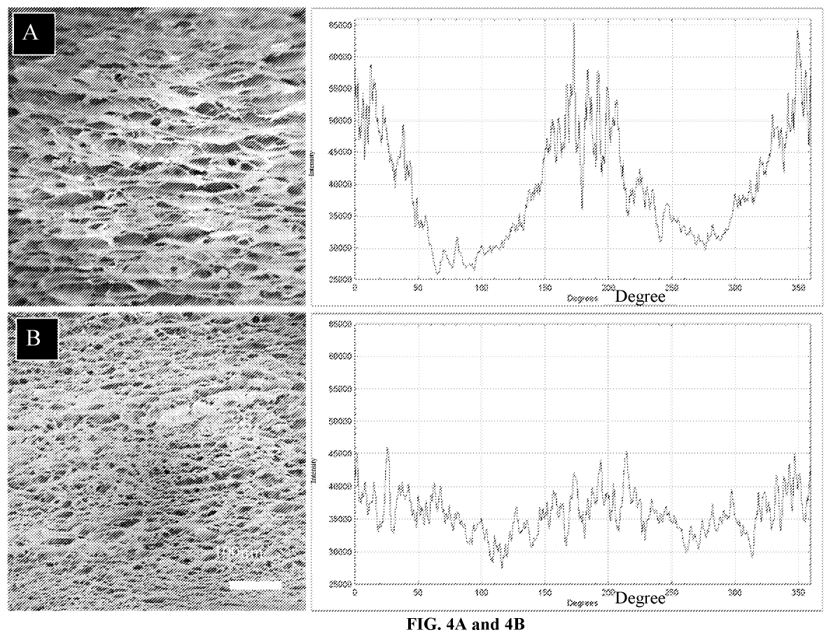

SEM (Auriga) micrographs were taken at Stevens Institute of Technology. The fiber orientation was measured from SEM micrographs using ImageJ. A radial summation of the pixel intensities between 0 and 360 degrees was obtained by an oval projection of fast Fourier transform (FFT) frequency (see FIG. 4). The percent of fiber oriented along the reference line was determined as the area occupied by the oriented fibers along the reference orientation. The percent of fiber oriented along the reference line was greater than 50%.

Biomechanical Properties

Tensile strength was determined from the Chatillon mechanical tester (Wilmington, N.C.). A uniform sample with a dimension of 5 mm×2 mm×10 mm (W×H×L) was cut. The sample was hydrated in 10 ml of water for 10 minutes and fixed at the bottom and top grips. It was pulled at a rate of 2.5 cm per minute until the sample breaks into two separate pieces. The ultimate tensile strength was 315 N/cm².

For suture retention strength, a double interrupted suture was used to simulate more closely to the clinical practice. A 5 mm distance was chosen in order to replicate the suggested distance in clinical practice. The suture loops were attached to a hook of the tester which attached to the load cell. The inner rim of the scaffold was clamped to the test stand (Chatillon Mechanical tester). The sample was pulled at a rate of 2.5 cm/minute. The suture pull-out strength was 9.4N.

### Hydrothermal Stability

Hydrothermal stability or hydrothermal shrinkage temperature (Tₛ) was determined from a differential scanning calorimeter (DSC, Mettler Toledo, Switzerland). The endothermic peak was defined as the shrinkage temperature, in ° C., of the specimen. The Tₛ was 72° C.

### Surface Friction Coefficient

The surface friction coefficient was determined from the in-house designed apparatus described previously. The apparatus simulated the motion of the knee joint. The number of vibrations of the pendulum per cycle defined the relative friction of the scaffold. The friction coefficient was calculated according to that published (Crisco, J J, et al., 2007, Proc. Inst. Mech. Eng. H, 221:325-333). The friction coefficient for collagen surface in PBS solution was about 0.21 (applied force 15 g). The friction coefficient for collagen surface in 0.2% HA solution was about 0.15 (applied force 15 g). This preliminary test indicated that a surface lubricated with HA significantly reduces the surface friction. The HA protection of the surface was demonstrated in FIG. 6.

### EXAMPLE

Biopolymeric materials were chosen for the fabrication of the extracellular matrix (ECM) implant. Among biopolymers, the use of type I collagen was preferred as type I collagen has been well accepted for the manufacture of resorbable implants for tissue repair over the past 30 years (Li, 2012). A special rotational device was designed for the engineer of a circumferentially oriented type I collagen fiber meniscus implant. FIG. 1a depicts the device 10. A rotational mandrel 11 is attached to a commercial digital stirrer 12 (Caframo Digital Stirrer, Georgian Bluffs, ON Canada). The speed of rotation can be controlled within the range of interest (1-50 RPM). At the top of the mandrel is a cone shaped stainless steel weight element 13 with a slight concave curvature to simulate the curvature of the meniscus. The weight element can slide along the mandrel once released from its fixed position by a pin (element 14). At the bottom of the device is a polycarbonate housing (element 15) having a dimension that is closely fit with the size of the weight element 13 and allow the weight to meet the bottom of the housing element 15. The bottom of the housing element was made permeable to water for facilitating dehydration.

A fixed weight of purified type I collagen fibers prepared in this laboratory was first suspended in a 0.07M lactic acid solution, pH 2.3 at 4° C. for overnight. The swollen collagen fibers were then further dispersed into fibrils with a commercial homogenizer (Silverson, East Longmeadow, Mass.). Upon adequate de-air by vacuum, the pH of the solution was adjusted to the isoelectric point of collagen (˜pH 5) to reconstitute the fibrils to macroscopic fibers. The fibers were then aligned with the rotational mandrel 11 with a speed between 1 RPM to 50 RPM. By this process all the macroscopic fibers were wound onto the mandrel. Upon partial dehydration of the collagen fibers with a glass plate while the mandrel was still rotating, the mandrel was then placed at the center of the housing element 15 (see FIG. 1a). The weight element 13 was then released from its fixed position by removing the pin 14 allowing weight element 13 to slide down to the top of the collagen fibers (FIG. 1b). The mandrel was then removed, and the weight was applied from the weight element to slowly dehydrate the collagen fibers (FIG. 1c). The dehydrated, molded, fiber-oriented meniscus scaffold was removed from the mold for freeze drying in a commercial freeze dryer (Virtis, Gardiner, N.Y.). The freeze-dried scaffold was then crosslinked with low molecular weight vapor aldehyde (formaldehyde or glycol-aldehyde). The final crosslinked collagen was cut along the center of the collagen matrix to provide two separate collagen menisci (FIG. 1d).

Different molecular weight hyaluronic acid (HA) molecules (0.1×10⁶ to 3×10⁶ Daltons) were then impregnated into the scaffold via needle injection. The total HA content was about 1-10% of the weight of the scaffold, preferably 2-7% of the weight of scaffold. The HA incorporated scaffold implant was then dried, packaged and sterilized.

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated by reference in their entireties.

What is claimed is:

1. A scaffold implant for repairing injured or diseased human meniscus, comprising fibers of a biopolymer impregnated with glycosaminoglycan, wherein greater than 50% of the biopolymer fibers are oriented along the circumferential direction; said scaffold implant comprising a density from 0.1 g/cm³ to 0.4 g/cm³; a pore volume from 60% to 90%; and a surface friction coefficient from 0.05 to 1.0.

2. The scaffold implant of claim 1, wherein the biopolymer is collagen.

3. The scaffold implant of claim 2, wherein the collagen is selected from the group consisting of type I, type II and type III collagens.

4. (canceled)

5. The scaffold implant of claim 3, wherein the collagen is type I collagen.

6. (canceled)

7. The scaffold implant of claim 1, wherein the glycosaminoglycan is hyaluronic acid, chondroitin sulfate, chitosan, or alginic acid, or a combination thereof.

8. (canceled)

9. The scaffold implant of claim 7, wherein the weight percent of the hyaluronic acid in the scaffold implant is in the range of about 1% to about 10% or about 2% to about 7%.

10. (canceled)

11. The scaffold implant of claim 8, wherein the molecular weight of the hyaluronic acid ranges from about 0.1×10⁶ Daltons to about 3.0×10⁶ Daltons.

12. The scaffold implant of claim 11, wherein hyaluronic acids of various molecular weights are impregnated into the scaffold implant via injection after the scaffold has been engineered.

13. The scaffold implant of claim 1, wherein greater than 55%, 60%, or 70% of the fibers are oriented along the circumferential direction.

14. (canceled)

15. (canceled)

16. The scaffold implant of claim 1, wherein the average density is from about 0.15 g/cm³ to about 0.25 g/cm³.

17. The scaffold implant of claim 1, wherein the friction coefficient is from about 0.08 to about 0.8.

18. The scaffold implant of claim 5, wherein the implant is crosslinked by aldehyde-based molecules.

19. (canceled)

20. The scaffold implant of claim 1, wherein the scaffold implant further contains one or more bioactive elements.

21. (canceled)

22. The scaffold implant of claim 20, wherein the bioactive elements include one or more selected from the group consisting of PRP, cells, and bioactive molecules.

23. The scaffold implant of claim 20, wherein the bioactive elements are allogenous and include bioactive molecules or stem cells.

24. The scaffold implant of claim 20, wherein the bioactive elements are autologous and include PRP, bioactive molecules and stem cells.

25. The scaffold implant of claim 21, wherein the bioactive elements are drugs.

26. A method for making a hyaluronic acid impregnated collagen-based scaffold implant comprising:
preparing a collagen dispersion;
reconstituting collagen from the collagen dispersion into fibers;
aligning the fibers onto a rotating mandrel to form aligned collagen fibers;
placing the aligned collagen fibers in a mold of defined dimension;
adding a weight to the fibers to dehydrate the fibers and form a dehydrated but still wet collagen scaffold matrix;
freeze drying the dehydrated but still wet collagen scaffold matrix to obtain a freeze-dried scaffold matrix;
crosslinking the freeze-dried scaffold matrix;
sizing the scaffold matrix; and
injecting hyaluronic molecules into the scaffold matrix.

27. A collagen-based meniscus scaffold implant prepared according to the method of claim 26.

28. A method of treating a joint of a subject, comprising:
providing a scaffold implant of claim 1; and
delivering the scaffold implant to the joint.
